(19) United States
(12) Patent Application Publication
(10) Pub. No.: US 2006/0198885 A1
(43) Pub. Date: Sep. 7, 2006

(54) Oral pharmaceutical composition

(75) Inventors: Nitin Bhalachandra Dharmadhikari, Mumbai (IN); Pankaj Khapra, Mumbai (IN)

Correspondence Address: MERCHANT & GOULD PC, P.O. BOX 2903, MINNEAPOLIS, MN, 55402-0903, US

(73) Assignee: Sun Pharmaceutical Industries Ltd., Mumbai (IN)

(21) Appl. No.: 11/359,053

(22) Filed: Feb. 22, 2006

(30) Foreign Application Priority Data
Feb. 22, 2005 (IN) 199/MUM/2005

(51) Int. Cl. A61K 31/495; A61K 9/20

(52) U.S. Cl. 424/464; 514/255.04

(57) ABSTRACT

An oral pharmaceutical composition having reduced bitterness comprising therapeutically effective amounts of cetirizine or its pharmaceutically acceptable salts, alkaline earth oxide and a pharmaceutically acceptable carrier comprising a disintegrant wherein the composition disintegrates rapidly in the oral cavity.

The present invention relates to an oral pharmaceutical composition having reduced bitterness comprising cetirizine or its pharmaceutically acceptable salts.

### BACKGROUND OF THE INVENTION

The active ingredients formulated into drug products often have an unpleasant taste. As the solubility of the active ingredient in saliva increases, so does its offensive taste. Conventionally, sweetners and flavorants have been used in taste-masking. These agents generally work by providing a secondary flavor to the composition, which overcomes the bitter taste. This technique is sometimes able to mask mildly bitter pharmaceuticals, but the traditional sweetners are not effective in masking the bitter flavour of powerfully bitter pharmaceutical agents such as cetirizine hydrochloride.

Use of ion exchange resins to mask the bitter taste of drugs has been explored extensively. For example, U.S. Pat. Nos. 5,413,782, 6,280,717, U.S. Patent Application No. 2002032245 disclose drug-ion exchange resin complexes wherein the drug is loaded onto the resin.

U.S. Pat. No. 6,455,533 claims a solid pharmaceutical composition for oral administration comprising a mixture of an active substance belonging to the substituted benzhydrylpiperazine family, optically active isomers thereof and at least one cyclodextrin, wherein said mixture does not contain any inclusion complexes.

Another approach of taste masking often explored is the use of polymer coating, i.e coating the drug particles or coating the dosage form. U.S. Pat. No. 5,876,759 claims a compressed dosage form comprising taste-masking coating comprising a blend of a cellulose acetate or cellulose acetate butyrate and a second polymer. United States Patent Application Number 20020119196 relates to chewable tablets containing texture masked particles comprising a core of drug; a first coating layer comprised of a taste masking agent that substantially covers the core; and a second coating layer on the surface of the first coating layer comprising a film forming polymer and an anti-grit agent. Such coating processes to mask the taste may be tedious, complex and time consuming. Moreover, such coating may hamper the release of the drug from the dosage form.

Other methods to mask taste include those disclosed in WO application 9959588 and U.S. Pat. No. 6,245,353, which use novel taste masking ingredients such as use of glycyrrhetic acid or its salt, use of organic edible acid and alkali or alkaline earth metal carbonate or bicarbonate. The disadvantage of these methods is that the products prepared by these methods may require additional packaging and storage specifications, since the formulations are effervescent in nature. Also, effervescent preparations provide a gritty mouth feel, which is not desirable.

Applicability of all above said approaches for taste masking varies from drug to drug and depends on the type of dosage form required. The ideal solution to bitterness reduction or inhibition is the discovery of a universal inhibitor of all the bitter-tasting substances that does not affect the other test modalities such as sweetners. But there is no single substance that acts as the univeral inhibitor of a bitter taste. [Harmik Sohi, Yasmin Sultana and Roop K. Khar; Drug Development and Industrial Pharmacy, vol. 30, No. 5, pp. 429-448, 2004]. Therefore, there is need to find effective method suitable for reducing the bitterness of cetirizine or its pharmaceutically acceptable salts.

### OBJECTS OF THE INVENTION

It is an object of the present invention to provide an oral pharmaceutical composition having reduced bitterness comprising cetirizine or its pharmaceutically acceptable salt.

It is another object of the present invention to provide an oral pharmaceutical composition having reduced bitterness comprising cetirizine or its pharmaceutically acceptable salt wherein the composition disintegrates rapidly in oral cavity.

### SUMMARY OF THE INVENTION

We have found an oral pharmaceutical composition having reduced bitterness comprising cetirizine or its pharmaceutically acceptable salt wherein the compostion disintegrates rapidly in the oral cavity.

Accordingly, the present invention provides an oral pharmaceutical composition having reduced bitterness comprising therapeutically effective amounts of cetirizine or its pharmaceutically acceptable salt, an alkaline earth oxide and a pharmaceutically acceptable carrier comprising a disintegrant, wherein the composition disintegrates rapidly in the oral cavity.

### DETAILED DESCRIPTION OF THE INVENTION

The present invention is directed to an oral pharmaceutical composition having reduced bitterness comprising therapeutically effective amounts of cetirizine or its pharmaceutically acceptable salt, an alkaline earth oxide and a pharmaceutically acceptable carrier comprising a disintegrant, wherein the composition disintegrates rapidly in the oral cavity.

Alkaline earth oxides such as magnesium oxide readily adsorb water and carbon dioxide on exposure to air. Moreover, because of their mineral origin, they have several impurities capable of catalyzing the decomposition of the drug substance (Jain G. et al, Indian Drugs, 1992, 29 page 453). Magnesium oxide has also been found to decrease the bioavailability of some drugs (Takahashi et al, J. Pharm. Sci., 1985; 74; 862 and Remon et al, Arzneimittel Forschung, 1983, 33 (1), page 117)). We have surprisingly found that alkaline earth oxide such as magnesium oxide are compatible with cetirizine or its pharmaceutically acceptable salt, do not reduce it's bioavailability but reduce it's bitter taste.

The term ‘reduced bitterness’ as used herein means that the oral composition is palatable and has satisfactory taste masking.

Now, the present invention in its various embodiments is described herein below.
a. An oral pharmaceutical composition having reduced bitterness comprising therapeutically effective amounts of cetirizine or its pharmaceutically acceptable salt, an alkaline earth oxide and a pharmaceutically acceptable carrier comprising a disintegrant wherein the composition disintegrates rapidly in the oral cavity.
b. An oral pharmaceutical composition as defined in (a) above, wherein the pharmaceutically acceptable salt of cetirizine is cetirizine hydrochloride.
c. An oral pharmaceutical composition as defined in (a) wherein alkaline earth oxide is magnesium oxide.
d. An oral pharmaceutical composition as defined in (c) wherein the magnesium oxide is used in the range of about 1 to about 5% by weight, based on the total weight of the composition.
e. An oral pharmaceutical composition as defined in (a) wherein the pharmaceutically acceptable carrier comprises one or more agents selected from binders, disintegrants, lubricants, flavourants, fillers and sweetners.
f. An oral pharmaceutical composition as defined in (e) wherein disintegrant is selected from a group comprising cross-linked polyvinylpyrolidone, croscarmellose sodium, calcium carboxyl methylcellulose, low substituted hydroxypropyl cellulose, alginic acid, sodium alginate, microcrystalline cellulose, sodium starch glycolate, and mixtures thereof.

Cetirizine or its pharmaceutically acceptable salt may be used in the present invention in therapeutically effective amounts. Preferably, cetirizine it is present as cetirizine hydrochloride an amount from about 1 mg to about 15 mg, particularly 5 mg and 10 mg.

The taste masking excipients of the present invention include alkaline earth oxides. The alkaline earth oxides may be used in an anhydrous form or in the form of their hydroxides. Examples of taste masking excipients that may be used in the present invention include magnesium oxide, calcium oxide, magnesium hydroxide and the like and mixtures thereof. The amount of taste masking excipients utilized ranges from about 0.1% to about 10%, preferably from about 1% to about 5% most preferably from about 2 to about 3% by weight of the composition. The amount of the alkaline earth oxide utilized is such that satisfactory taste masking is achieved, while not compromising on the disintegration of the composition in the oral cavity.

In a preferred embodiment of the present invention, magnesium oxide is used as the taste masking excipients. In particular, the magnesium oxide that may be used in the present invention may be either in a bulky, namely light form or in a dense form, termed commonly as heavy. The USP defines heavy as, 15 g occupies a volume of about 30 ml and for a light variety, 20 grams occupy a volume of about 150 ml. Both these forms occur as fine, while, odourless powders. They possess a cubic crystal structure. The light magnesium oxide shows a particle size distribution of 99.8% less than 45 μm in size.

The oral pharmaceutical composition of the present invention comprises pharmaceutically acceptable carrier comprising one or more disintegrants such that the composition disintegrates rapidly in the oral cavity. The disintegrants may be selected from the group comprising cross-linked polyvinylpyrolidone, croscarmellose sodium, calcium carboxylmethylcellulose, low substituted hydroxypropyl cellulose, alginic acid, sodium alginate, microcrystalline cellulose, sodium starch glycolate, and the like and mixtures thereof. The amount of the disintegrant utilized is such that the composition of the present invention disintegrates rapidly. Typically, the disintegrants may be used in an amount ranging from about 0.1% to about 90% w/w of the composition, more particularly, in an amount ranging from about 5% to about 50% w/w of the composition, most preferably from about 15% to about 40% by weight of the composition. The disintegration time may vary from about less than 3 minute, preferably less than about 2 minutes; most preferably less than 1 minute when placed in the oral cavity.

Sweetners are used to impart a pleasant flavour to the taste masked composition. Preferred sweetners include artifical sweetners such as aspartame, saccharin, cyclamates, mixtures thereof and the like. Natural sweetners such as sucrose, fructose, sodium glycolate and other mono- and disaccharides are also preferred. The sweetner is used in an amount ranging from about 0.02% to about 5% by weight of the oral composition of the present invention.

Flavourants may also be used to improve the flavor of the composition. The flavourants may be used singly or in combination. Preferred flavourants include, but are not limited to, cherry, strawberry, grape, cream, vanilla, choclate, trutti fruty, mocha, spearmint, cola and the like. In general, the total amount of flavourant required to elicit safisfactory flavoring of the composition is not more than 3% by weight of the pharmaceutical composition.

The pharmaceutical excipients that may be used in the pharmaceutical carrier of the present invention are conventional excipients and are used in amounts conventionally used in the pharmaceutical art. The oral pharmaceutical composition of the present invention may be obtained by wet granulation or dry granulation or direct compession processes, known to a person of skill in the art. In granulation process, it is preferable to mix the alkali earth oxide with the cetirizine or its pharmaceutically acceptable salt and granulate. The granules then may be mixed with extragranular excipients and compressed into a tablet.

The invention is further illustrated by the following examples, which are by no means intended to limit the scope of the invention but are given by way of illustration.

### EXAMPLE 1

A composition of the present invention is prepared according to the formula as described below.

TABLE 1

Quantity

| Ingredients | mg/tab | % w/w |
|---|---|---|
| Cetirizine hydrochloride | 10.0 | 2.5 |
| Magnesium oxide (heavy) | 10.0 | 2.5 |
| Sodium starch glycolate (Type A) | 90.0 | 22.5 |
| Pregelatinized Starch | 20.0 | 5.0 |
| Microcrystalline cellulose with guar gum | 161.0 | 40.25 |
| (Avicel CE 15) | | |
| Microcrystalline cellulose (Avicel PH 102) | 88.1 | 22.05 |
| FD&C Blue No. 2 | 2.3 | 0.575 |
| FD&C Red No. 40 | 1.1 | 0.275 |
| Acesulfame potassium | 8.0 | 2.00 |
| Tuttifrutti flavour | 2.0 | 0.5 |
| Prosweet | 1.5 | 0.375 |
| Magnesium stearate | 2.0 | 0.50 |
| Talc | 2.0 | 0.50 |
| Colloidal silicon dioxide | 2.0 | 0.50 |

Cetirizine hydrochloride, magnesium oxide and sodium starch glycolate were sifted separately, dry mixed and granulated with a suitable quantity of water. The milled wet granules were dried at 60° C. (55-65° C.) and further milled. The milled granules were mixed with sifted Avicel PH 102, Starch 1500 and Avicel CE 15. These dried granules were mixed with the sweeteners, flavourants, lubricants and the blend was subjected to compression.

The tablets were found to have a hardness of 4.2-5.3 Kp with a disintegration time of about 2 minutes and 101% drug released in 15 minutes. These tablets were found to have a satisfactory taste.

### EXAMPLE 2

A composition of the present invention is prepared according to the formula as described below.

TABLE 2

Quantity

| Ingredients | mg/tab | % w/w |
|---|---|---|
| Cetirizine hydrochloride | 5.0 | 2.5 |
| Magnesium oxide (heavy) | 5.0 | 2.5 |
| Sodium starch glycolate (Type A) | 45.0 | 22.5 |
| Pregelatinized Starch (starch 1500) | 100 | 5.0 |
| Microcrystalline cellulose with | 80.5 | 40.25 |
| guar gum (Avicel CE 15) | | |
| Microcrystalline cellulose (Avicel PH 102) | 44.05 | 22.05 |
| FD&C Blue No. 2 | 1.15 | 0.575 |
| FD&C Red No. 40 | 0.55 | 0.275 |
| Acesulfame potassium | 4.0 | 2.00 |
| Tuttifrutti flavour | 1.0 | 0.5 |
| Prosweet | 0.75 | 0.375 |
| Magnesium stearate | 1.0 | 0.50 |
| Talc | 1.0 | 0.50 |
| Colloidal silicon dioxide | 1.0 | 0.50 |

Cetirizine hydrochloride, magnesium oxide and sodium starch glycolate were sifted separately, dry mixed and granulated with a suitable quantity of water. The milled wet granules were dried at 60° C. (55-65° C.) and further milled. The milled granules were mixed with sifted Avicel PH 102, Starch 1500 and Avicel CE 15. These dried granules were mixed with the sweeteners, flavourants, lubricants and the blend was subjected to compression.

The tablets were found to have a hardness of 2.1-2.7 Kp with a disintegration time of about 55 seconds and 99% drug released in 15 minutes. These tablets were found to have a satisfactory taste.

### COMPARATIVE EXAMPLE 1

A comparative example was prepared according to the following formula.

TABLE 3

| | Quantity | |
|---|---|---|
| Ingredients | mg/tablet | % w/w |
| Cetirizine hydrochloride | 10.0 | 2.7 |
| Sodium starch glycolate (Type A) | 100.0 | 26.8 |
| Pregelatinized Starch (starch 1500) | 20.0 | 5.4 |
| Microcrystalline cellulose with guar gum (Avicel | 160.85 | 43.2 |
| CE 15) | | |
| Microcrystalline cellulose (Avicel PH 102) | 93.75 | 25.2 |
| Acesulfame potassium | 4.4 | 1.18 |
| Peppermint flavour | 2.0 | 0.53 |
| Prosweet | 1.5 | 0.4 |
| Magnesium stearate | 2.0 | 0.53 |
| Talc | 2.0 | 0.53 |
| Colloidal silicon dioxide | 2.0 | 0.53 |

Cetirizine hydrochloride and sodium starch glycolate was sifted separately, dry mixed and granulated with a suitable quantity of water. The granules thus obtained were milled and dried at 60° C. and further milled. The milled granules were mixed with sifted Avicel PH 102, Starch 1500 and Avicel CE 15. These dried granules were mixed with the sweeteners, flavourants, lubricants and the blend was subjected to compression.

The tablets so prepared showed a hardness of 3.5 Kp, with disintegration time 9 seconds. The composition showed a desirable disintegration time and dissolution profile, but the taste masking was not effective, indicating that use of a alkaline oxide, such as magnesium oxide used in Example 1 above, is essential for masking the bitter taste of cetirizine.

### COMPARATIVE EXAMPLE 2

A tablet of cetirizine was obtained as described below.

TABLE 4

| | Quantity | |
|---|---|---|
| Ingredients | mg/tablet | % w/w |
| Cetirizine Hydrochloride and magnesium oxide | 110 | 27.5 |
| granules | | |
| Acesulfame potassium | 7.0 | 1.75 |
| Microcrystalline cellulose and guar gum | 255.0 | 63.75 |
| (Avicel CE 15) | | |
| Pregelatinized starch ((starch 1500) | 20.0 | 5.0 |
| Colloidal silicon dioxide | 2.0 | 0.5 |
| Peppermint flavour | 2.0 | 0.5 |
| Talc | 2.0 | 0.5 |
| Magnesium stearate | 2.0 | 0.5 |

Cetirizine hydrochloride was blended with magnesium oxide in the ratio of 1:10. The blend was then wet granulated with purified water. The granules so obtained were mixed with the other ingredients and compressed. Although the tablets gave satisfactory taste masking effect, the dissolution profile showed that only 83% of cetirizine hydrochloride was released in 30 minutes, thus indicating that use of disintegrant(s) such as sodium starch glycolate, is essential for causing rapid disintegration and complete release of the drug from the tablet.

While the invention has been described by reference to specific embodiments, this was done for purposes of illustration only and should not be construed to limit the spirit or the scope of the invention.

1. An oral pharmaceutical composition having reduced bitterness comprising therapeutically effective amounts of cetirizine or its pharmaceutically acceptable salts, an alkaline earth oxide and a pharmaceutically acceptable carrier wherein the composition disintegrates rapidly in the oral cavity.

2. An oral pharmaceutical composition as in claim 1 wherein the alkaline earth oxide is selected from the group comprising calcium oxide, magnesium oxide and mixtures thereof.

3. An oral pharmaceutical composition as in claim 1 wherein amount of alkaline earth oxide ranges from about 0.1% to about 10% by weight of the composition.

4. An oral pharmaceutical composition as in claim 3 wherein the amount of alkaline earth oxide ranges from about 1% to about 5% by weight of the composition.

5. An oral pharmaceutical composition as in claim 1 wherein the disintegrant is selected from the group comprising of cross-linked polyvinylpyrolidone, croscarmellose sodium, calcium carboxylmethylcellulose, low substituted hydroxypropyl cellulose, alginic acid, sodium alginate, microcrystalline cellulose, sodium starch glycolate.

6. An oral pharmaceutical composition as in claim 5 wherein the amount of disintegrant ranges from about 15% to about 40% by weight of the composition

7. An oral pharmaceutical composition as in claim 1 wherein cetirizine or its pharmaceutically acceptable salt may be present in an amount ranging from about 1 mg to about 15 mg.

8. An oral pharmaceutical composition as in claim 1 wherein the composition disintegrates rapidly in the oral cavity in about 1 minute to about 2 minutes.

9. An oral pharmaceutical composition as in claim 1 wherein the composition disintegrates rapidly in oral cavity in less than 1 minute.
